(19) United States
(12) Patent Application Publication
(10) Pub. No.: US 2017/0336416 A1
(43) Pub. Date: Nov. 23, 2017

(54) COMPOSITIONS FOR ENHANCING CHEMILUMINESCENCE AND METHODS OF USING THE SAME

(71) Applicant: Abbott Laboratories, Abbott Park, IL (US)

(72) Inventor: Jonathan Grote, Libertyville, IL (US)

(21) Appl. No.: 15/626,904

(22) Filed: Jun. 19, 2017

Related U.S. Application Data
(62) Division of application No. 14/278,700, filed on May 15, 2014, now abandoned.

(51) Int. Cl. G01N 33/58; C07D 219/04

(52) U.S. Cl. CPC G01N 33/582; C07D 219/04

(57) ABSTRACT

Disclosed herein is a composition for enhancing chemiluminescence and a method of using the composition to improve analyte detection.

### TECHNICAL FIELD

The present invention relates to a composition for enhancing chemiluminescence and a method of using the composition to improve analyte detection.

### BACKGROUND

Chemiluminescence is a chemical reaction involving a compound and an oxidant (e.g., a peroxide compound). Particularly, a molecular reorganization occurs in which the O—O bond in the peroxide compound is cleaved or fragmented in the presence of alkaline conditions, thereby releasing energy. The released energy is absorbed by the compound to give an excited state intermediate, wherein the intermediate compound emits light (i.e., a chemiluminescent signal) as it decays to its ground state. Suitable compounds include acridinium esters and acridinium sulfonamides, which have been used extensively in a variety of diagnostic methods to facilitate the detection of an analyte in a sample. Such methods, for example, directly link the acridinium compound to a binding partner of the analyte or an analyte analog and relate the presence or an amount of the analyte in the sample to the measured chemiluminescent signal.

Characteristics of the chemiluminescent signal such as the emission wavelength, the duration of emission, and the intensity (total light emission) are affected by the structure of the acridinium compound, the pH of the solution in which the chemiluminescent reaction occurs, and the presence of other reagents during the chemiluminescent reaction. In turn, these characteristics can limit the detection capabilities of the diagnostic methods. Limitations may include sensitivity, accuracy, and the range of concentrations over which the analyte may be detected.

Accordingly, a need exists for the identification and development of new compositions that increase total light emission from a chemiluminescent reaction, and thus, enhance the chemiluminescent signal. Furthermore, the generation of the enhanced chemiluminescent signal must be compatible with diagnostic methods so as to improve the sensitivity, dynamic range, and accuracy of these methods.

### SUMMARY OF THE INVENTION

The present invention is directed to a composition comprising: (a) a compound of formula (I)

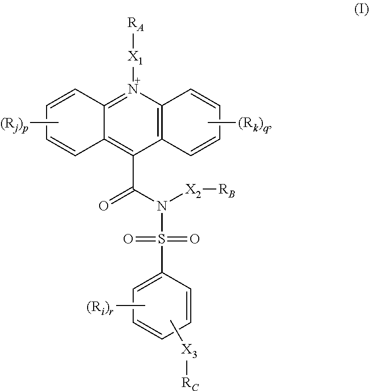

wherein

X₁, X₂ and X₃ are each independently selected from a bond and alkylene;

one of R_{A} and R_{B} is —SO₃⁻;

the other of R_{A} and R_{B} is selected from the group consisting of hydrogen, alkyl, alkenyl, alkynyl, aryl, amino, amido, alkoxy, hydroxyl, carboxy, halogen, halide, nitro, cyano, —SO₃H and —C(O)R^{1a};

R_{C} is —C(O)R^{2a} or H;

Rⱼ, Rₖ and Rᵢ, at each occurrence, are independently selected from the group consisting of hydrogen, alkyl, alkenyl, alkynyl, aryl, arylalkyl, amino, amido, acyl, alkoxy, hydroxyl, carboxy, halogen, halide, nitro, cyano, —SO₃H, —SO₃⁻, sulfoalkyl, carboxyalkyl and acylalkyl;

p, q and r are each independently 0, 1, 2, 3 or 4; and

R^{1a} and R^{2a}, at each occurrence, are independently selected from the group consisting of hydrogen, alkyl, alkoxy, haloalkyl, haloalkyloxy, hydroxyl, cycloalkyl, cycloalkyloxy, cycloalkylalkyl, aryl, aryloxy, arylalkyl, halogen, heterocycle, heterocycleoxy, heterocyclealkyl, heteroaryl, heteroaryloxy and heteroarylalkyl; and

(b) at least one zwitterionic detergent.

In the above composition, X₃ may be alkylene; one of R_{A} and R_{B} may be —SO₃⁻; and the other of R_{A} and R_{B} may be selected from the group consisting of alkyl, hydroxyl, carboxy, —SO₃H and —C(O)R^{1a}.

In the above composition, X₃ may be —CH₂—CH₂—CH₂—; and R^{2a} may be selected from the group consisting of hydroxyl, aryloxy, heterocycleoxy and heteroaryloxy.

In the above composition, X₁ and X₂ may be alkylene; one of R_{A} and R_{B} may be —SO₃⁻; and the other of R_{A} and R_{B} may be —SO₃H.

In the above composition, R^{2a} may be selected from the group consisting of hydroxyl, 4-nitrophenoxy, perchlorophenoxy, perfluorophenoxy, 2,5-dioxopyrrolidin-1-yloxy, 1,3-dioxoisoindolin-2-yloxy, and benzotriazol-1-yloxy.

In the above composition, the compound of Formula (I) may be selected from the group consisting of: 3-(9-(((4-(3-carboxypropyl)phenyl)sulfonyl)(3-sulfopropyl)carbamoyl)acridin-10-ium-10-yl)propane-1-sulfonate; 3-(9-((3-carboxypropyl)(tosyl)carbamoyl)acridin-10-ium-10-yl)propane-1-sulfonate; 3-(9-(((4-(4-oxo-4-(perfluorophenoxy)butyl)phenyl)sulfonyl)(3-sulfopropyl)carbamoyl)acridin-10-ium-10-yl)propane-1-sulfonate; 3-(N-((4-(3-carboxypropyl)phenyl)sulfonyl)-10-(3-sulfopropyl)acridin-10-ium-9-carboxamido)propane-1-sulfonate; and 3-(N-((4-(4-oxo-4-(perfluorophenoxy)butyl)phenyl)sulfonyl)-10-(3-sulfopropyl)acridin-10-ium-9-carboxamido)propane-1-sulfonate.

In the above composition, the at least one zwitterionic detergent may be present in an amount sufficient to form micelles. In the above composition, the at least one zwitterionic detergent may be N-dodecyl-N,N-dimethyl-3-ammonio-1-propanesulfonate, 3-(Tetradecyldimethylammonio)propanesulfonate, or a combination thereof The above composition may further comprise an oxidant or a basic solution. The above composition may enhance a chemiluminescence signal about 1.5-fold to about 20-fold.

The present invention is also directed to a kit for producing a chemiluminescent signal. The kit may comprise the above composition. In the kit, the at least one zwitterionic detergent may be N-dodecyl-N,N-dimethyl-3-ammonio-1-propanesulfonate, 3-(Tetradecyldimethylammonio)propanesulfonate, or a combination thereof In the kit, the compound of Formula (I) may be 3-(9-(((4-(3-carboxypropyl)phenyl)sulfonyl)(3-sulfopropyl)carbamoyl)acridin-10-ium-10-yl)propane-1-sulfonate or 3-(9-((3-carboxypropyl)(tosyl)carbamoyl)acridin-10-ium-10-yl)propane-1-sulfonate. In the kit, the compound of formula (I) may be conjugated to an agent. The agent may be selected from the group consisting of a polynucleotide, a polypeptide, and an analog of an analyte.

The present invention is also directed to a method for detecting an analyte in a test sample. The method may comprise contacting the sample with the above composition. The method may further comprise generating a chemiluminescent signal and measuring the chemiluminescent signal. The chemiluminescent signal may be related to an amount of the analyte in the test sample.

In the above method, the analyte may be detected by an immunoassay.

The present invention is also directed to a composition comprising 3-(9-(((4-(3-carboxypropyl)phenyl)sulfonyl)(3-sulfopropyl)carbamoyl)acridin-10-ium-10-yl)propane-1-sulfonate, 3-(9-((3-carboxypropyl)(tosyl)carbamoyl)acridin-10-ium-10-yl)propane-1-sulfonate, or a combination thereof.

The present invention is also directed to a compound of formula (I)

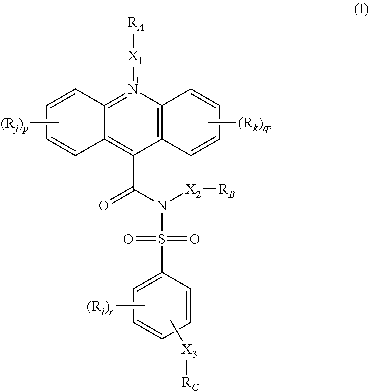

wherein

X₁, X₂ and X₃ are each independently selected from a bond and alkylene;

one of R_{A} and R_{B} is —SO₃⁻;

the other of R_{A} and R_{B} is selected from the group consisting of hydrogen, alkyl, alkenyl, alkynyl, aryl, amino, amido, alkoxy, hydroxyl, carboxy, halogen, halide, nitro, cyano, —SO₃H and —C(O)R^{1a};

R_{C} is —C(O)R^{2a} or H;

Rⱼ, Rₖ and Rᵢ, at each occurrence, are independently selected from the group consisting of hydrogen, alkyl, alkenyl, alkynyl, aryl, arylalkyl, amino, amido, acyl, alkoxy, hydroxyl, carboxy, halogen, halide, nitro, cyano, —SO₃H, —SO₃⁻, sulfoalkyl, carboxyalkyl and acylalkyl;

p, q and r are each independently 0, 1, 2, 3 or 4; and

R^{1a} and R^{2a}, at each occurrence, are independently selected from the group consisting of hydrogen, alkyl, alkoxy, haloalkyl, haloalkyloxy, hydroxyl, cycloalkyl, cycloalkyloxy, cycloalkylalkyl, aryl, aryloxy, arylalkyl, halogen, heterocycle, heterocycleoxy, heterocyclealkyl, heteroaryl, heteroaryloxy and heteroarylalkyl.

In the above compound, X₃ may be alkylene; one of R_{A} and R_{B} may be —SO₃⁻; and the other of R_{A} and R_{B} may be selected from the group consisting of alkyl, hydroxyl, carboxy, —SO₃H and —C(O)R^{1a}.

In the above compound, X₃ may be —CH₂—CH₂—CH₂—; and R^{2a} may be selected from the group consisting of hydroxyl, aryloxy, heterocycleoxy and heteroaryloxy.

In the above compound, X₁ and X₂ may be alkylene; one of R_{A} and R_{B} may be —SO₃⁻; and the other of R_{A} and R_{B} may be —SO₃H.

In the above compound, R^{2a} may be selected from the group consisting of hydroxyl, 4-nitrophenoxy, perchlorophenoxy, perfluorophenoxy, 2,5-dioxopyrrolidin-1-yloxy, 1,3-dioxoisoindolin-2-yloxy, and benzotriazol-1-yloxy.

The above compound may be selected from the group consisting of: 3-(9-(((4-(3-carboxypropyl)phenyl)sulfonyl)(3-sulfopropyl)carbamoyl)acridin-10-ium-10-yl)propane-1-sulfonate; 3-(9-((3-carboxypropyl)(tosyl)carbamoyl)acridin-10-ium-10-yl)propane-1-sulfonate; 3-(9-(((4-(4-oxo-4-(perfluorophenoxy)butyl)phenyl)sulfonyl)(3-sulfopropyl)carbamoyl)acridin-10-ium-10-yl)propane-1-sulfonate; 3-(N-((4-(3-carboxypropyl)phenyl)sulfonyl)-10-(3-sulfopropyl)acridin-10-ium-9-carboxamido)propane-1-sulfonate; and 3-(N-((4-(4-oxo-4-(perfluorophenoxy)butyl)phenyl)sulfonyl)-10-(3-sulfopropyl)acridin-10-ium-9-carboxamido)propane-1-sulfonate.

### BRIEF DESCRIPTION OF THE DRAWINGS

FIG. 1 shows a graph plotting reaction vessel (RV) versus relative light unit (RLU). The solid bars represented the results with Triton X-100 and the cross-hatched bars represented the results with N-dodecyl-N,N-dimethyl-3-ammonio-1-propanesulfonate (under the trade name ZWITTERGENT 3-12).

FIG. 2 shows a graph plotting reaction vessel (RV) versus relative light unit (RLU). The solid bars represented the results with Triton X-100 and the cross-hatched bars represented the results with N-dodecyl-N,N-dimethyl-3-ammonio-1-propanesulfonate (under the trade name ZWITTERGENT 3-12).

FIG. 3 shows a graph plotting reaction vessel (RV) verus relative light unit (RLU). The dark solid bars represented the results with Triton X-100. The light solid bars represented results with 0.6 mM 3-(Tetradecyldimethylammonio)propanesulfonate (under the tradename ZWITTERGENT 3-14) in both solutions. The cross-hatched bars represented results with 6 mM and 0.6 mM 3-(Tetradecyldimethylammonio)propanesulfonate (under the tradename ZWITTERGENT 3-14) in the hydrogen peroxide and sodium hydroxide solutions, respectively.

### DETAILED DESCRIPTION

The present invention relates to a composition comprising an acridinium based compound and a zwitterionic detergent. The acridinium based compound may be a compound of formula (I). The acridinium based compound is also zwitterionic and interacts with the zwitterionic detergent to enhance a chemiluminescent signal. The zwitterionic detergent may be present in an amount sufficient to reach critical micelle concentrations (CMCs). Critical micelle concentrations of the zwitterionic detergent enhance the chemiluminescent signal of the composition. For example, the chemiluminescent signal of the composition is enhanced about 1.5-fold to about 20-fold or about 50% to about 2000% over the chemiluminescent signal generated by compositions comprising acridinium sulfonamides and non-zwitterionic detergents. In another example, the chemiluminescent signal of the composition is enhanced about 1.5-fold to about 20-fold or about 50% to about 2000% over the chemiluminescent signal generated by compositions, in which the zwitterionic detergent is replaced with a non-zwitterionic detergent.

The chemiluminescent signal of the composition is enhanced because the chemiluminescent reaction releases energy within the hydrophobic interiors of the micelles formed by the zwitterionic detergent. These hydrophobic interiors exclude water. Water attenuates or reduces the release of energy, and thus, the chemiluminescent signal. Accordingly, the hydrophobic interiors of the zwitterionic detergent micelles isolate the release of energy from water; and thus, the energy release is not attenuated or reduced by the presence of water. This isolation of energy release away from water results in an enhanced chemiluminescent signal.

Additionally, the chemiluminescent signal of the composition is enhanced because of favorable electronic and/or ionic interactions between the acridinium based compound and the zwitterionic detergent. The interactions between the zwitterionic detergent and the acridinium based compound lower the activation energy required for the chemiluminescent reaction (i.e., the reaction between the acridinium based compound and an oxidant) to proceed, thereby leading to significantly and unexpectedly enhanced chemiluminescent signals due to faster reaction rates as compared to rates achievable with acridinium sulfonamides and non-zwitterionic detergents.

The concentration of the zwittergent detergent in the composition may alter or influence the total light emission from the chemiluminescent reaction. Accordingly, the zwittergent detergent may be used within the composition to manipulate or control light emission the chemiluminescent reaction.

The enhanced chemiluminescence generated by the composition significantly increases the total light emission from the chemiluminescent reaction, and therefore, the ratio of signal to background noise (signal to noise ratio) is more distinguishable. A greater signal to noise ratio provides more accurate measurement of the chemiluminescent signal and a larger dynamic range for distinguishing differences between measurements (i.e., increased sensitivity). Therefore, the present invention provides an advantage in applications where sensitivity is important, such as immunoassays and hybridization assays, because smaller amounts of analyte can be detected, analyte is more consistently detected, and the range of concentration over which the analyte is detected is larger.

The present invention also relates to a method of using the composition to improve the detection of the analyte.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “activation energy”, as used herein, refers to the minimum amount of energy that must be added to a chemical system for a reaction to occur.

The term “acyl” as used herein, refers to a —C(O)Rₐ group where Rₐ is hydrogen, alkyl, cycloalkyl, cycloalkylalkyl, halogen, aryl or arylalkyl. Representative examples of acyl include, but are not limited to, formyl, acetyl, cyclohexylcarbonyl, cyclohexylmethylcarbonyl, benzoyl, benzylcarbonyl and the like.

The term “acylalkyl” as used herein, refers to an acyl group, as defined herein, attached to the parent molecular moiety through an alkyl group, as defined herein.

The term “alkenyl” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxyl” or “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkylene” as used herein, refers to a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkynyl” as used herein, refers to a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “amido” as used herein, refers to an amino group attached to the parent molecular moiety through a carbonyl group.

The term “amino” as used herein, refers to —NR_{b}R_{c}, wherein R_{b} and R_{c}, at each occurrence, are independently selected from the group consisting of hydrogen, alkyl and acyl.

As used herein, the term “analyte” refers to any substance for which exists a naturally occurring binding member (or agent) or for which a binding member (or agent) may be prepared. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), virus particles and metabolites of or antibodies to any of the above substances.

The term “aryl” as used herein, refers to a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl. The aryl groups of the present disclosure can be optionally substituted with one, two, three, four, or five substituents independently selected from the group consisting of alkoxy, alkyl, carboxyl, halo, and hydroxyl.

The term “arylalkyl” as used herein, refers to an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryloxy” as used herein, refers to an aryl group attached to the parent molecular moiety through an oxy group, as defined herein.

The term “carbonyl”, as used herein, refers to a —C(O)— group.

The term “carboxyl” or “carboxy” as used herein, refers to —CO₂H or —CO₂;

The term “carboxyalkyl” as used herein, refers to a carboxy group attached to the parent molecular moiety through an alkyl group, as defined herein.

The term “critical micelle concentration”, as used herein, refers to the concentration of surfactants (e.g., detergents) necessary for micelles to form. One skilled in the art could easily determine the amount of detergent needed to reach critical micelle concentration in the composition.

The term “cyano” as used herein, refers to a —CN group.

The term “cycloalkyl” as used herein, refers to a saturated monocyclic, bicyclic, or tricyclic hydrocarbon ring system having 3 to 12 carbon atoms. Representative examples of cycloalkyl groups include cyclopropyl, cyclopentyl, bicycle[3.1.1]heptyl, adamantyl, and the like. The cycloalkyl groups of the present disclosure can be optionally substituted with one, two, three, four, or five substituents independently selected from the group consisting of alkoxy, alkyl, carboxyl, halo, and hydroxyl.

The term “cycloalkylalkyl” as used herein, refers to a cycloalkyl group attached to the parent molecular moiety through an alkyl group, as defined herein.

The term “cycloalkyloxy” as used herein, refers to a cycloalkyl group attached to the parent molecular moiety through an oxy group, as defined herein.

As used herein, the term “enhance” refers to increasing the total light emission of a chemiluminescent reaction and/or the signal to background noise ratio of the chemiluminescent reaction.

The term “excited state”, as used herein, refers to any energy state higher than the ground state.

The term “ground state”, as used herein, refers to the lowest energy state of a system.

The term “haloalkyl” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkyloxy” as used herein, refers to a haloalkyl group attached to the parent molecular moiety through an oxy group, as defined herein.

The term “halogen” as used herein, refers to a —Cl, —Br, —I, or —F.

The term “halide” as used herein, refers to a binary compound, of which one part is a halogen atom and the other part is an element or radical that is less electronegative than the halogen, e.g., an alkyl radical.

The term “heteroaryl” as used herein, refers to an aromatic monocyclic ring or an aromatic bicyclic ring system. The aromatic monocyclic rings are five- or six-membered rings containing at least one heteroatom independently selected from the group consisting of N, O and S. The five-membered aromatic monocyclic rings have two double bonds and the six-membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, benzimidazolyl, benzofuranyl, benzothiazolyl, benzothienyl, benzoxazolyl, furyl, imidazolyl, indazolyl, indolyl, indolizinyl, isobenzofuranyl, isoindolyl, isoxazolyl, isoquinolinyl, isothiazolyl, naphthyridinyl, oxadiazolyl, oxazolyl, phthalazinyl, pyridinyl, pyridazinyl, pyrimidinyl, pyrazinyl, pyrazolyl, pyrrolyl, quinolinyl, quinolizinyl, quinoxalinyl, quinazolinyl, tetrazolyl, thiadiazolyl, thiazolyl, thienyl, triazolyl and triazinyl. The heteroaryl groups of the present disclosure may be optionally substituted with 1, 2, or 3 substituents independently selected from the group consisting of alkoxy, alkyl, carboxyl, halo, and hydroxyl.

The term “heteroarylalkyl” as used herein, refers to a heteroaryl group, as defined herein, attached to the parent molecular moiety through an alkyl group, as defined herein.

The term “heteroaryloxy” as used herein, refers to a heteroaryl group attached to the parent molecular moiety through an oxy group, as defined herein.

The term “heterocycle” as used herein, refers to a non-aromatic monocyclic ring or a non-aromatic bicyclic ring. The non-aromatic monocyclic ring is a three, four, five, six, seven, or eight membered ring containing at least one heteroatom, independently selected from the group consisting of N, O and S. Representative examples of monocyclic ring systems include, but are not limited to azetidinyl, aziridinyl, diazepinyl, dithianyl, imidazolinyl, imidazolidinyl, isothiazolinyl, isoxazolinyl, isoxazolidinyl, morpholinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydro-2H-pyranyl, tetrahydro-2H-pyran-2-yl, tetrahydro-2H-pyran-4-yl, tetrahydrothienyl, thiazolinyl, thiazolidinyl, thiomorpholinyl, 1,1-dioxidothiomorpholinyl (thiomorpholine sulfone) and thiopyranyl. The bicyclic heterocycles are exemplified by a monocyclic heterocycle appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Bicyclic ring systems are also exemplified by a bridged monocyclic ring system in which two non-adjacent atoms of the monocyclic ring are linked by a bridge of between one and three additional atoms selected from the group consisting of carbon, nitrogen and oxygen. Representative examples of bicyclic ring systems include, but are not limited to, for example, benzopyranyl, benzothiopyranyl, benzodioxinyl, 1,3-benzodioxolyl, cinnolinyl, 1,5-diazocanyl, 3,9-diaza-bicyclo[4.2.1]non-9-yl, 3,7-diazabicyclo[3.3.1]nonane, octahydro-pyrrolo[3,4-c]pyrrole, indolinyl, isoindolinyl, 2,3,4,5-tetrahydro-1H-benzo[c]azepine, 2,3,4,5-tetrahydro-1H-benzo[b]azepine, 2,3,4,5-tetrahydro-1H-benzo[d]azepine, tetrahydroisoquinolinyl and tetrahydroquinolinyl. The heterocycle groups of the present disclosure may be optionally substituted with 1, 2, or 3 substituents independently selected from the group consisting of alkoxy, alkyl, carboxyl, halo, and hydroxyl.

The term “heterocyclealkyl” as used herein, refers to a heterocycle group, as defined herein, attached to the parent molecular moiety through an alkyl group, as defined herein.

The term “heterocycleoxy” as used herein, refers to a heterocycle group attached to the parent molecular moiety through an oxy group, as defined herein.

The term “hydroxyl” or “hydroxy” as used herein, refers to an —OH group.

As used herein, the term “microparticle” refers to any material which is insoluble, or can be made insoluble by a subsequent reaction and is in a particulate form. Thus, microparticles can be latex, plastic, derivatized plastic, magnetic or non-magnetic metal, glass, silicon, or the like. A vast array of microparticle configurations are also well known and include, but are not limited to, beads, shavings, grains, or other particles, well known to those skilled in the art. Microparticles according to the present invention preferably are between 0.1 micron and 1 micron in size and more preferably between 0.3 micron and 0.6 micron in size.

The term “nitro” as used herein, refers to an —NO₂ group.

The term “oxy” as used herein, refers to an —O— group.

As used herein, the term “polynucleotide(s)” refers to a polymer of DNA or RNA, modified DNA or RNA, DNA or RNA mimetics or nucleic acid analogs such as uncharged nucleic acid analogs including, but not limited to, peptide nucleic acids (PNAs) which are disclosed in International Publication No. WO 92/20702 or morpholino analogs which are described in U.S. Pat. Nos. 5,185,444; 5,034,506; and 5,142,047, all of which are herein incorporated by reference. As used herein, the term “polynucleotide” includes polynucleotides composed of naturally-occurring nucleobases, sugars, and covalent internucleoside (backbone) linkages as well as polynucleotides having non-naturally-occurring portions which function similarly. Polynucleotides can be routinely synthesized using a variety of techniques that are currently available. For example, such sequences can be synthesized using conventional nucleotide phosphoramidite chemistry and the instruments available from Applied Biosystems, Inc. (Foster City, Calif.); DuPont (Wilmington, Del.); or Milligen (Bedford, Mass.). It will be understood, however, that sequences employed as primers should at least comprise DNA at the 3′ end of the sequence and preferably are completely comprised of DNA.

The term “sulfoalkyl” as used herein, refers to a —SO₃H or —SO₃⁻ group attached to the parent molecular moiety through an alkyl group, as defined herein.

As used herein, the term “specific binding member” refers to a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

As used herein, the term “solid support” refers to any material which is insoluble, or can be made insoluble by a subsequent reaction. Thus, a solid support can be latex, plastic, derivatized plastic, magnetic or non-magnetic metal, glass, silicon, or the like. A vast array of solid support configurations are also well known and include, but are not limited to, beads, shavings, grains, particles, plates, or tubes.

As used herein, the term “target sequence” refers to a polynucleotide sequence that is detected, amplified, both amplified and detected or otherwise complementary to a polynucleotide sequence conjugated to the compound of formula (I) as herein provided. While the term target sequence is sometimes referred to as singled stranded, those skilled in the art will recognize that the target sequence may actually be double stranded.

As used herein, the term “test sample” generally refers to a biological material being tested for and/or suspected of containing an analyte of interest. The biological material may be derived from any biological source. Examples of biological materials include, but are not limited to, stool, whole blood, serum, plasma, red blood cells, platelets, interstitial fluid, salvia, ocular lens fluid, cerebral spinal fluid, sweat, urine, ascites fluid, mucous, nasal fluid, sputum, synovial fluid, peritoneal fluid, vaginal fluid, menses, amniotic fluid, semen, or soil, fermentation broths cell cultures, chemical reaction mixtures and the like. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, lysing, etc. If such methods of pretreatment are employed with respect to the test sample, such pretreatment methods are such that the analyte of interest remains in the test sample at a concentration proportional to that in an untreated test sample (e.g., namely, a test sample that is not subjected to any such pretreatment method(s)).

The term “zwitterion”, as used herein, refers to a net neutral molecule that has both a positive and negative charge.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 1. COMPOSITION

Provided herein is a composition comprising an acridinium based compound and a zwitterionic detergent. The acridinium based compound may be a compound of formula (I). The acridinium based compound is also zwitterionic and interacts with the zwitterionic detergent to enhance a chemiluminescent signal. The zwitterionic detergent may be present in an amount sufficient to reach critical micelle concentrations (CMCs). Critical micelle concentrations of the zwitterionic detergent may enhance the chemiluminescent signal of the composition.

The chemiluminescent signal of the composition is enhanced because the chemiluminescent reaction releases energy within the hydrophobic interiors of the micelles formed by the zwitterionic detergent. These hydrophobic interiors exclude water. Water attenuates or reduces the release of energy, and thus, the chemiluminescent signal. Accordingly, the hydrophobic interiors of the zwitterionic detergent micelles isolate the release of energy from water, and thus, energy release is not attenuated or reduced by the presence of water. This isolation of energy release away from water results in an enhanced chemiluminescent signal.

The chemiluminescent signal is also enhanced because of favorable electronic and/or ionic interactions between the compound of formula (I) and the zwitterionic detergent. The interactions between the zwitterionic detergent and the acridinium based compound lower the activation energy required for the chemiluminescent reaction (i.e., the reaction between the acridinium based compound and an oxidant) to proceed, thereby leading to significantly and unexpectedly enhanced chemiluminescent signals due to faster reaction rates as compared to rates achievable with acridinium sulfonamides and non-zwitterionic detergents.

The composition may enhance the chemiluminescent signal about 1.5 to about 20-fold, or about 1.5 to about 10-fold, over chemiluminescent signals generated by compositions comprising acridinium sulfonamides and non-zwitterionic detergents. The composition may enhance the chemiluminescent signal about 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20-fold. The composition may enhance the chemiluminescent signal about 5-fold or about 10-fold.

The composition may enhance the chemiluminescent signal about 1.5 to about 20-fold, or about 1.5 to about 10-fold, over chemiluminescent signals generated by compositions, in which the zwitterionic detergent is replaced with a non-zwitterionic detergent. The composition may enhance the chemiluminescent signal about 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20-fold. The composition may enhance the chemiluminescent signal about 5-fold or about 10-fold.

The composition may also enhance the chemiluminescent signal about 50% to about 2000%, or about 50% to about 1000%, over chemiluminescent signals generated by compositions comprising acridinium sulfonamides and non-zwitterionic detergents. The composition may enhance the chemiluminescent signal about 50%, 100%, 150%, 200%, 250%, 300%, 350%, 400%, 450%, 500%, 550%, 600%, 650%, 700%, 750%, 800%, 850%, 900%, 950%, 1000%, 1050%, 1100%, 1150%, 1200%, 1250%, 1300%, 1350%, 1400%, 1450%, 1500%, 1550%, 1600%, 1650%, 1700%, 1750%, 1800%, 1850%, 1900%, 1950% or 2000%.

The composition may also enhance the chemiluminescent signal about 50% to about 2000%, or about 50% to about 1000%, over chemiluminescent signals generated by compositions, in which the zwitterionic detergent is replaced with a non-zwitterionic detergent. The composition may enhance the chemiluminescent signal about 50%, 100%, 150%, 200%, 250%, 300%, 350%, 400%, 450%, 500%, 550%, 600%, 650%, 700%, 750%, 800%, 850%, 900%, 950%, 1000%, 1050%, 1100%, 1150%, 1200%, 1250%, 1300%, 1350%, 1400%, 1450%, 1500%, 1550%, 1600%, 1650%, 1700%, 1750%, 1800%, 1850%, 1900%, 1950% or 2000%.

The enhanced chemiluminescence generated by the composition significantly increases the total light emission from the chemiluminescent reaction, thereby increasing the chemiluminescent signal to noise ratio. The increased signal to noise ratio may be about 1.5:1 to about 20:1, or about 1.5:1 to about 10:1. The increased signal to noise ratio may also be about 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, 15:1, 15.5:1, 16:1, 16.5:1, 17:1, 17.5:1, 18:1, 18.5:1, 19:1, 19.5:1, or 20:1.

The concentration of the zwittergent detergent in the composition may alter or influence the total light emission from the chemiluminescent reaction. Accordingly, the zwittergent detergent may be used within the composition to manipulate or control light emission the chemiluminescent reaction.

The composition may further comprise an oxidant (i.e., an oxidizing agent) and a base (i.e., basic or alkaline solution). The base reacts with the oxidant to increase the reactivity of the oxidant. The oxidant then reacts with the compound of formula (I) to produce an “excited state” acridone derivative. The excited state acridone derivative releases energy in the form of light (i.e., chemiluminescence) as it decays to a “ground state” acridone derivative. As described above, this release of energy can occur within the hydrophobic interiors of the zwitterionic detergent micelles, which isolate the release of energy from water; and thus, the energy release is not attenuated or reduced by the presence of water. This isolation of energy release away from water results in an enhanced chemiluminescent signal.

The chemiluminescence generated in the above described reaction can be detected using routine techniques known to those skilled in the art, as described in more detail below.

The compound of formula (I) may also be linked or conjugated to an agent to form a conjugate. The compound of formula (I) and the agent may be linked through a covalent bond. Methods of linking the compound of formula (I) and the agent are known to those skilled in the art. For example, the compound of formula (I) and the agent together may form an amide or ester moiety, as described below.

a. Compounds of Formula I

The composition comprises an acridinium based compound. The acridinium based compound may be a compound of formula (I). The compound of formula (I) is zwitterionic in nature, and has a positive charge on the acridinium ring and a negative charge on an —SO₃⁻ group. The compound of formula (I) may interact with the zwitterionic detergent to enhance the chemiluminescent signal. Specifically, such an interaction may be electronic and/or ionic. The interactions between the zwitterionic detergent and the acridinium based compound lower the activation energy required for the chemiluminescent reaction (i.e., the reaction between the acridinium based compound and an oxidant) to proceed, thereby leading to significantly and unexpectedly enhanced chemiluminescent signals due to faster reaction rates as compared to rates achievable with known acridinium sulfonamides and non-zwitterionic detergents. The compound of formula (I) may be

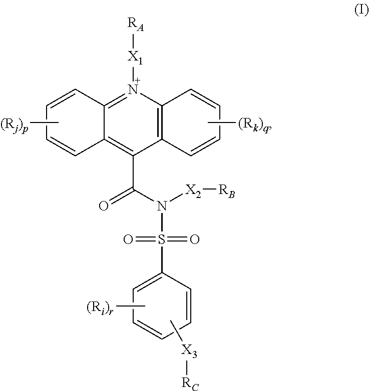

wherein,

X₁, X₂ and X₃ are each independently selected from a bond and alkylene;

one of R_{A} and R_{B} is —SO₃⁻;

the other of R_{A} and R_{B} is selected from the group consisting of hydrogen, alkyl, alkenyl, alkynyl, aryl, amino, amido, alkoxy, hydroxyl, carboxyl, halogen, halide, nitro, cyano, —SO₃H and —C(O)R^{1a};

R_{C} is —C(O)R^{2a} or H;

Rⱼ, Rₖ and Rᵢ, at each occurrence, are independently selected from the group consisting of hydrogen, alkyl, alkenyl, alkynyl, aryl, arylalkyl, amino, amido, acyl, alkoxyl, hydroxyl, carboxyl, halogen, halide, nitro, cyano, —SO₃H, —SO₃⁻, sulfoalkyl, carboxyalkyl and acylalkyl;

p, q and r are each independently 0, 1, 2, 3 or 4; and

R^{1a} and R^{2a}, at each occurrence, are independently selected from the group consisting of hydrogen, alkyl, alkoxy, haloalkyl, haloalkyloxy, hydroxyl, cycloalkyl, cycloalkyloxy, cycloalkylalkyl, aryl, aryloxy, arylalkyl, halogen, heterocycle, heterocycleoxy, heterocyclealkyl, heteroaryl, heteroaryloxy and heteroarylalkyl.

The acridinium sulfonamide compound may be a compound of formula (I), wherein X₁, X₂, R_{C}, Rᵢ, Rⱼ, Rₖ, p, q, R^{1a} and R^{2a} are as described above;

X₃ is alkylene;

one of R_{A} and R_{B} is —SO₃⁻; and

the other of R_{A} and R_{B} is selected from the group consisting of alkyl, hydroxyl, carboxyl, —SO₃H and —C(O)R^{1a}.

The acridinium sulfonamide compound may be a compound of formula (I), wherein X₁, X₂, R_{A}, R_{B}, R_{C}, Rᵢ, Rⱼ, Rₖ, p, q, and R^{1a} are as described above;

X₃ is —CH₂—CH₂—CH₂—; and

R^{2a} is selected from the group consisting of hydroxyl, aryloxy, heterocycleoxy and heteroaryloxy.

The acridinium sulfonamide compound may be a compound of formula (I), wherein X₃, R_{C}, Rᵢ, Rⱼ, Rₖ, p, q, R^{1a} and R^{2a} are as described above;

X₁ and X₂ are alkylene;

one of R_{A} and R_{B} is —SO₃⁻; and

the other of R_{A} and R_{B} is —SO₃H.

The acridinium sulfonamide compound may be a compound of formula (I), wherein X₁, X₂, X₃, R_{A}, R_{B}, R_{C}, Rᵢ, Rⱼ, Rₖ, p, q, and R^{1a} are as described above; and

R^{2a} is selected from the group consisting of hydroxyl, 4-nitrophenoxy, perchlorophenoxy, perfluorophenoxy, 2,5-dioxopyrrolidin-1-yloxy, 1,3-dioxoisoindolin-2-yloxy, and benzotriazol-1-yloxy.

The chemiluminescent compounds may be a compound of formula (I) selected from the group consisting of:

3-(9-(((4-(3-carboxypropyl)phenyl)sulfonyl)(3-sulfopropyl)carbamoyl)acridin-10-ium-10-yl)propane-1-sulfonate;

3-(9-((3-carboxypropyl)(tosyl)carbamoyl)acridin-10-ium-10-yl)propane-1-sulfonate;

3-(9-(((4-(4-oxo-4-(perfluorophenoxy)butyl)phenyl)sulfonyl)(3-sulfopropyl)carbamoyl)acridin-10-ium-10-yl)propane-1-sulfonate;

3-(N-((4-(3-carboxypropyl)phenyl)sulfonyl)-10-(3-sulfopropyl)acridin-10-ium-9-carboxamido)propane-1-sulfonate; and

3-(N-((4-(4-oxo-4-(perfluorophenoxy)butyl)phenyl)sulfonyl)-10-(3-sulfopropyl)acridin-10-ium-9-carboxamido)propane-1-sulfonate.

The chemiluminescent compounds may be a compound of formula (I) selected from the group consisting of:

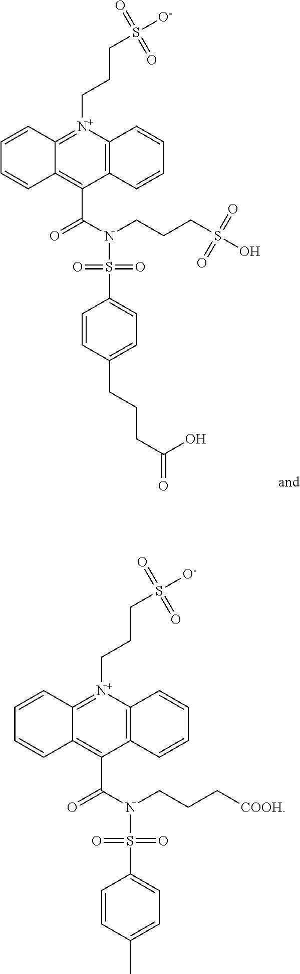 [SMILES: O=C(O)CCCc1ccc(S(=O)(=O)N(CCCS(=O)(=O)O)C(=O)c2c3ccccc3[n+](CCCS(=O)(=O)[O-])c3ccccc23)cc1 ; Cc1ccc(S(=O)(=O)N(CCCC(=O)O)C(=O)c2c3ccccc3[n+](CCCS(=O)(=O)[O-])c3ccccc23)cc1]

As described above, the compound of formula (I) is zwitterionic because it comprises a positively charged acridinium ring, and a negatively charged —SO₃⁻ group. The —SO₃⁻ substituent is located at the R_{A} or R_{B} position while the positive charge is located at the nitrogen of the acridinium ring.

The compound of formula (I) may comprise a substituent group that is suitable for linking to the agent. Particularly, the R_{C} group may be a substituent that can react with the agent to form the conjugate. An R_{C} group of formula (I) that may be suitable for linking to the agent includes, but is not limited to, a carboxylic acid or an ester. Such an R_{C} group, together with a suitable agent, may form an ester or amide moiety, as described below.

b. Detergents

The composition further comprises at least one zwitterionic detergent. The zwitterionic detergent enhances the chemiluminescent signal generated by the composition by forming micelles. These micelles have hydrophobic interiors, which exclude water. Energy release by the chemiluminescent reaction can occur within these hydrophobic interiors, and thus, is protected from water, which attenuates or reduces energy release.

The zwitterionic detergent also enhances the chemiluminescent signal generated by the composition through favorable electronic and/or ionic interactions with the acridinium based compound Particularly, interactions between the zwitterionic detergent and the acridinium based compound lower the activation energy required for the chemiluminescent reaction (i.e., the reaction between the acridinium based compound and an oxidant) to proceed. Thus, the zwitterionic detergent facilitates faster reactions between the acridinium based compound and the oxidant, wherein faster reactions release more energy (i.e., emit a larger, or more intense chemiluminescent signal) than a composition comprising a acridinium sulfonamide and a non-zwitterionic detergent.

The zwitterionic detergent may be present in an amount sufficient to reach critical micelle concentrations (CMCs). Critical micelle concentrations of the zwitterionic detergent may enhance the chemiluminescent signal of the composition by increasing the reaction rate for the above described chemiluminescent reaction and isolating energy release by the chemiluminescent reaction from water as described above.

The concentration of the zwittergent detergent in the composition may alter or influence the total light emission from the chemiluminescent reaction. Accordingly, the zwittergent detergent may be used within the composition to manipulate or control light emission the chemiluminescent reaction.

The composition may comprise the zwitterionic detergent in a range from about 0.1% to about 20% by weight, or about 0.25% to about 5% by weight, based on the total weight of the composition. The zwitterionic detergent may also be present in about 0.25%, 0.5%, 1%, 1.5%, 2%, 2.5%, 3%, 3.5%, 4%, 4.5% or 5%, based on the total weight of the composition. The zwitterionic detergent may also be present in the composition at a concentration of about 0.1 mM to about 100 mM, about 0.1 mM to about 50 mM, about 1 mM to about 100 mM, about 1 mM to about 50 mM, about 0.6 mM, about 4.3 mM, about 6 mM or about 32 mM. The zwitterionic detergent may be present in the composition at a concentration of 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2mM, 2.1 mM, 2.2 mM, 2.3 mM, 2.4 mM, 2.5 mM, 2.6 mM, 2.7 mM, 2.8 mM, 2.9 mM, 3mM, 3.1 mM, 3.2 mM, 3.3 mM, 3.4 mM, 3.5 mM, 3.6 mM, 3.7 mM, 3.8 mM, 3.9 mM, 4mM, 4.1 mM, 4.2 mM, 4.3 mM, 4.4 mM, 4.5 mM, 4.6 mM, 4.7 mM, 4.8 mM, 4.9 mM, 5mM, 5.1 mM, 5.2 mM, 5.3 mM, 5.4 mM, 5.5 mM, 5.6 mM, 5.7 mM, 5.8 mM, 5.9 mM, 6mM, 6.1 mM, 6.2 mM, 6.3 mM, 6.4 mM, 6.5 mM, 6.6 mM, 6.7 mM, 6.8 mM, 6.9 mM, 7mM, 7.1 mM, 7.2 mM, 7.3 mM, 7.4 mM, 7.5 mM, 7.6 mM, 7.7 mM, 7.8 mM, 7.9 mM, 8mM, 8.1 mM, 8.2 mM, 8.3 mM, 8.4 mM, 8.5 mM, 8.6 mM, 8.7 mM, 8.8 mM, 8.9 mM, 9mM, 9.1 mM, 9.2 mM, 9.3 mM, 9.4 mM, 9.5 mM, 9.6 mM, 9.7 mM, 9.8 mM, 9.9 mM, 10mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM.

The zwitterionic detergent may be 1-dodecanoyl-sn-glycero-3-phosphocholine, 3-(4-tert-butyl-1-pyridinio)-1-propanesulfonate, 3-(N,N-dimethylmyristylammonio) propanesulfonate, 3-(1-pyridinio)-1-propanesulfonate, 3-(benzyldimethylammonio) propanesulfonate, 3-(decyldimethylammonio)propanesulfonate, 4-{N,N-Dimethyl-N-[3-(tetradecanoylamino)propyl]ammonio}butanesulfonate (ASB 14-4), 3-[N,N-Dimethyl(3-myristoylaminopropyl)ammonio]propanesulfonate (ASB-14), 3-{N,N-Dimethyl-N-[3-(4-octylbenzoylamino)propyl]ammonio}propanesulfonate (ASB-C80), 3-[(3-Cholamidopropyl)dimethylammonio]-1-propanesulfonate (CHAPS), CHAPS hydrate, 3-([3-Cholamidopropyl] dimethylammonio)-2-hydroxy-1-propanesulfonate (CHAPSO), N-Dodecyl-N,N-(dimethylammonio)butyrate (DDMAB), dimethylethylammoniumpropane sulfonate, N,N-Dimethyl-N-dodecylglycine betaine (EMPIGEN® BB detergent), miltefosine, miltefosine hydrate, N,N-dimethyldodecylamine N-oxide, N-dodecyl-N,N-dimethyl-3-ammonio-1-propanesulfonate (under the tradename ZWITTERGENT 3-12), O-(decylphosphoryl)choline, O-(octylphosphoryl)choline, poly(maleic anhydride-alt-1-decene), 3-(dimethylamino)-1-propylamine, 3-(Decyldimethylazaniumyl)propane-1-sulfonate (under the tradename ZWITTERGENT 3-10), 3-(Tetradecyldimethylammonio)propanesulfonate (under the tradename ZWITTERGENT 3-14), 3-(N,N-Dimethylpalmitylammonio)propanesulfonate (under the tradename ZWITTERGENT 3-16), and sodium 2,3-dimercaptopropanesulfonate monohydrate, or combinations thereof.

c. Oxidant

The composition may further comprise an oxidant. The oxidant reacts with the base to increase the reactivity of the oxidant. The oxidant then reacts with the acridinium based compound to produce an “excited state” acridone derivative, wherein the excited state acridone derivative releases energy in the form of light (i.e., chemiluminescence) as it decays to a “ground state” acridone derivative. The oxidant may be hydrogen peroxide or an enzyme that produces a peroxide. One skilled in the art could easily determine the amount of oxidant to be used in the method described below. For example, the amount of oxidant that can be added to the composition may be from about 0.0001 unit/mL to about 10,000 units/mL. The oxidant may be present in the composition from about 0.025% (v/v) to about 2.5% (v/v). The oxidant may be present in the composition from about 0.025% (v/v), 0.050% (v/v), 0.075% (v/v), 0.10% (v/v), 0.20% (v/v), 0.30% (v/v), 0.40% (v/v), 0.50% (v/v), 0.60% (v/v), 0.70% (v/v), 0.80% (v/v), 0.90% (v/v), 1.0% (v/v), 1.1% (v/v), 1.2% (v/v), 1.3% (v/v), 1.4% (v/v), 1.5% (v/v), 1.6% (v/v), 1.7% (v/v), 1.8% (v/v), 1.9% (v/v), 2.0% (v/v), 2.1% (v/v), 2.2% (v/v), 2.3% (v/v), 2.4% (v/v) or 2.5% (v/v).

The peroxide producing enzyme may be selected from the group consisting of (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

d. Basic Solution

The composition may further comprise a basic or alkaline solution that contains a base. The base reacts with the oxidant as described above to produce a peroxide anion (i.e., a more reactive oxidant), wherein the peroxide anion reacts with the acridinium based compound to generate a chemiluminescent signal, as described above.

The basic solution may have a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate or a combination thereof. One skilled in the art could easily determine the amount of basic solution to be used in the method described below.

e. Conjugates

The acridinium based compound, as part of the composition described above, may be further linked or conjugated to an agent to form a conjugate. The acridinium based compound and the agent may be linked through a covalent bond (e.g., the compound and agent together may form an amide or ester moiety). This conjugate may be used in the methods described below to detect an analyte.

(1) Moieties

The conjugate may include the acridinium based compound and the agent together forming an ester or amide moiety. Methods of forming these moieties are known to those skilled in the art. For example, conjugates comprising ester moieties may be formed by reacting a compound, for example a compound of formula (I), where R_{C} is —C(O)R^{2a} and R^{2a} is aryloxy, heterocycleoxy or heteroaryloxy, preferably R^{2a} is 4-nitrophenoxy, perchlorophenoxy, perfluorophenoxy, 2,5-dioxopyrrolidin-1-yloxy 1,3-dioxoisoindolin-2-yloxy, and benzotriazol-1-yloxy with an agent comprising at least one hydroxy group. Conjugates comprising ester moieties may also be formed by reacting a compound of formula (I), wherein R_{C} is —C(O)R^{2a} and R^{2a} is hydroxyl, with an agent comprising at least one hydroxy group, optionally in the presence of a coupling agent, optionally in the presence of a co-reagent, such as 1-hydroxybenzotriazole (HOBt), and optionally in the presence of a catalyst, such as dimethylaminopyridine (DMAP).

Conjugates comprising amide moieties may be formed by reacting a compound of formula (I), where R_{C} is —C(O)R^{2a} and R^{2a} is aryloxy, heterocycleoxy or heteroaryloxy, preferably R^{2a} is 4-nitrophenoxy, perchlorophenoxy, perfluorophenoxy, 2,5-dioxopyrrolidin-1-yloxy, 1,3-dioxoisoindolin-2-yloxy, and benzotriazol-1-yloxy with an agent comprising at least one amino group. Conjugates comprising amide moieties may also be formed by reacting a compound of formula (I), wherein R_{C} is —C(O)R^{2a} and R^{2a} is hydroxyl, with an agent comprising at least one amino group, optionally in the presence of a coupling agent, optionally in the presence of a co-reagent, such as 1-hydroxybenzotriazole (HOBt), and optionally in the presence of a catalyst, such as dimethylaminopyridine (DMAP).

Suitable coupling agents include, but are not limited to, dicyclohexylcarbodiimide (DCC), diisopropylcarbodiimide (DIC), ethyl-(N′,N′-dimethylamino)propylcarbodiimide hydrochloride (EDC), benzotriazol-1-yloxy)tris(dimethylamino)phosphonium hexafluorophosphate (BOP), benzotriazol-1-yl-oxytripyrrolidinophosphonium hexafluorophosphate (PyBOP), O-(benzotriazol-1-yl)-N,N,N′,N′-tetramethyluronium hexafluorophosphate (HBTU), O-(benzotriazol-lyl)-N,N,N′,N′-tetramethyluronium tetrafluoroborate (TBTU), O-(7-Azabenzotraizol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), O-(3,4-dihydro-4-oxo-1,2,3-benzotriazine-3-yl)-N,N,N′,N′-tetramethyluronium tetrafluoroborate (TDBTU), and 3-(diethylphosphoryloxy)-1,2,3-benzotriazin-4(3H)-one (DEPBT).

(2) Agents

The conjugate may include at least one hydroxy and/or at least one amino group of the agent forming an ester or amide moiety with the acridinium based compound. The agent may bind an analyte in a test sample, thereby relating the chemiluminescent signal to an amount of the analyte in the test sample, as described in the method below. Alternatively, the agent may compete with the analyte in the test sample for binding to a binding partner of the analyte.

The agent may be selected from alkaloids, amino acids, antigens, bacteria, cDNA, drugs (including those administered for therapeutic purposes as well as those administered for illicit purposes), drug intermediaries or byproducts, haptens, hormones, lipids, medicaments, microorganisms, nucleic acids including nucleotides (deoxyribonucleotides and ribonucleotides), DNA (deoxyribonucleic acids), PNA (peptide nucleic acids), RNA (ribonucleic acids), single and double stranded nucleic acids, natural and synthetic nucleic acids, oligonucleotides, organic compounds, peptides, polynucleotides, proteins, sugars, steroids, virus particles, vitamins, toxins and metabolites of or antibodies to any of the above substances.

### 2. KIT

Provided herein is a kit that includes the composition and can be utilized in the method described below to generate the chemiluminescent signal. The kit may also include one or more containers for holding the composition. The kit may further include other material(s), which may be desirable from a user standpoint, such as a buffer(s), a diluent(s), a standard(s), and/or any other material useful in sample processing, washing, or conducting any other step of the method described herein.

The kit according to the present disclosure preferably includes instructions for carrying out the method of the invention. Instructions included in the kit of the present disclosure may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

### 3. METHOD

Also provided herein is a method of detecting an analyte in a test sample. The method includes contacting the test sample with the composition to form a mixture. The time and order in which the acridinium based compound, oxidant, basic solution, and zwitterionic detergent are added to the test sample is not critical. Additionally, the mixture formed by the composition and the test sample can optionally be allowed to incubate for a period of time. For example, the mixture can be allowed to incubate for a period of time from about 1 second to about 60 minutes. Specifically, the mixture can be allowed to incubate for a period from about 1 second to about 18 minutes.

Optionally, the test sample may be treated prior to the addition of any one or more of acridinium based compound, oxidant, basic solution, and zwitterionic detergent. Such treatment may include dilution, ultrafiltration, extraction, precipitation, dialysis, chromatography, and digestion. Moreover, if such treatment methods are employed with respect to the test sample, such treatment methods have analyte that remains in the test sample at a concentration proportional to that in an untreated test sample (e.g., namely, a test sample that is not subjected to any such treatment method(s)).

The method further includes measuring the chemiluminescent signal generated by the addition of the acridinium based compound, oxidant, basic solution, and zwitterionic detergent to the test sample. The signal generated by the mixture of the composition and the test sample is detected for a fixed duration of time. Preferably, the mixture is formed and the signal is detected concurrently. The duration of detection may range from about 0.01 second to about 360 seconds, more preferably from about 0.1 to about 30 seconds, and most preferably from about 0.5 to about 5 seconds. Chemiluminescent signals generated can be detected using routine techniques known to those skilled in the art, for example, luminometers, x-ray film, high speed photographic film, a CCD camera, and so forth. In some embodiments, detection may be semi-automated or automated.

The amount or intensity of the chemiluminescent signal generated can be used to quantify an amount of the analyte in the test sample. Specifically, the amount of the analyte in the test sample is proportional to the intensity of the signal generated. In alternative embodiments, the amount of the analyte in the test sample may be inversely proportional to the intensity of the signal generated. The amount of the analyte can be quantified by comparing the amount of light generated to a standard curve for the analyte or by comparison to a reference standard. The standard curve can be generated using serial dilutions or solutions of known concentrations of the analyte by mass spectrometry, gravimetric methods, and other techniques known in the art.

It may be desirable to include a control sample. The control sample may be analyzed concurrently with the test sample. The results obtained from the test sample can be compared to the results obtained from the control sample.

The method may be used in any number of applications in which an analyte is detected, for example, an immunoassay and assays employing nucleic acid amplification and/or hybridization. In such applications, the acridinium based compound may be conjugated to an agent as described above. The method improves the detection of the analyte in these applications because, as described above, the composition significantly increases the total light emission from the chemiluminescent reaction. In turn, the increase in total light emission provides a larger dynamic range for distinguishing differences between measurements, a greater signal to noise ratio, and more consistent measurements. Applications which employ the present invention gain the advantageous properties of detecting smaller amounts of the analyte (i.e., improved sensitivity), more consistent detection of the analyte (i.e., improved accuracy), a larger range of concentrations over which the analyte may be detected, and greater discernment of differences in the amount of the analyte between test samples (i.e., also improved sensitivity).

a. Immunoassay

The method may be used in an immunoassay, for example, a sandwich assay or a competitive binding assay. The immunoassay may use one or more antibodies or fragments thereof that specifically bind to the analyte as described in more detail below.

(1) Sandwich Assay

The immunoassay may be the sandwich assay, which measures the amount of the analyte between two layers of antibodies (i.e., at least one capture antibody and at least one detection antibody). The capture antibody and detection antibody are employed to separate and quantify the analyte in the test sample. Either monoclonal or polyclonal antibodies may be used for the capture and detection antibodies in the sandwich assay.

The capture antibody and detection antibody bind to different epitopes on the analyte. Desirably, binding of the capture antibody to an epitope on the analyte does not interfere with binding of the detection antibody to another epitope on the analyte. In the sandwich assay, the binding of the capture antibody to its epitope is not diminished by the binding of the detection antibody to its epitope.

The capture antibody may be used to capture the analyte in the test sample while the detection antibody may be the agent described above, and thus, is conjugated to the compound of formula (I). Accordingly, the detection antibody translates the amount of the analyte within the test sample into a measurable chemiluminescent signal.

Particularly, the capture antibody and detection antibody, when added to the test sample, bind the analyte to form a capture antibody-analyte-detection antibody complex. The capture antibody and detection antibody may be used in molar excess amounts relative to the maximum amount of the analyte expected in the test sample. Any capture antibody and/or detection antibody not bound to the analyte may be separated from the capture antibody-analyte-detection antibody complexes using routine techniques known to those skilled in the art.

The oxidant, basic solution, and zwitterionic detergent may be added to the separated complexes. Upon formation of the composition, the chemiluminescent signal is generated and then detected. The measured intensity of the chemiluminescent signal is compared to the reference standard and/or standard curve to quantify the amount of the analyte in the test sample.

In some embodiments, the capture antibody may be added to the test sample being assessed for the analyte to form a first mixture, in which the capture antibody and any analyte in the test sample form a capture antibody-analyte complex. After formation of the capture antibody-analyte complexes, any unbound capture antibody can be removed or separated from this first mixture using any technique known in the art, for example, but not limited to, washing. After removal of the unbound capture antibody, the detection antibody may be added to the first mixture to form a second mixture, in which the capture antibody-analyte-detection antibody complex is formed. Then as described above, any detection antibody not bound to the analyte may be separated from the capture antibody-analyte-detection antibody complexes, followed by formation of the composition and generation and detection of the chemiluminescent signal.

In other embodiments, the capture antibody and detection antibody may be simultaneously added the test sample to form the capture antibody-analyte-detection antibody complexes. Any capture antibody and/or detection antibody not bound to the analyte may be separated from the capture antibody-analyte-detection antibody complexes, followed by formation of the composition and generation and detection of the chemiluminescent signal.

In still other embodiments, the detection antibody may be added to the test sample before the capture antibody to form detection antibody-analyte complexes, which in turn, form capture antibody-analyte-detection antibody complexes upon addition of the capture antibody. Any capture antibody and/or detection antibody not bound to the analyte may be separated from the capture antibody-analyte-detection antibody complexes, followed by formation of the composition and generation and detection of the chemiluminescent signal.

(a) Capture Antibody

As described above, capture antibody may be used to capture the analyte in the test sample. In some embodiments, the capture antibody may be immobilized on a solid support or a microparticle to facilitate separation of the capture antibody-analyte-detection antibody complexes and/or capture antibody-analyte complexes from the test sample prior to or concurrently with detection of the chemiluminescent signal.

Suitable methods for immobilizing the capture antibody on the solid support or microparticle include ionic, hydrophobic, covalent interactions, and the like. The solid support or microparticle can be chosen for its intrinsic ability to attract and immobilize the capture antibody. Alternatively, the solid support or microparticle may comprise an additional receptor that has the ability to attract and immobilize the capture antibody. The additional receptor may include a charged substance that is oppositely charged with respect to the capture antibody, which is immobilized (attached to) the solid support or microparticle and has the ability to immobilize the capture antibody through a specific binding reaction. The additional receptor enables the indirect binding of the capture antibody to the solid support or microparticle before the performance of the method or during the performance of the method.

(2) Competitive Binding Assay

The immunoassay may be the competitive binding assay, which correlates the amount of the analyte in the test sample relative to the agent (e.g., analyte or an analog of the analyte (i.e., analyte-analog)), which is conjugated to the compound of formula (I) to form the conjugate as described above. The competitive binding assay may also include one or more antibodies that specifically bind to the analyte and conjugate (via the analyte or analyte-analog). Accordingly, in the competitive binding assay, the analyte and conjugate compete for binding to the antibody, and thus, the concentration of the analyte in the test sample determines the amount of the conjugate that will specifically bind to the antibody. The amount of the analyte present in the test sample is inversely proportional to the amount of conjugate bound to the antibody.

In the competitive binding assay, the antibody and conjugate are added to the test sample to form a mixture, in which analyte-antibody and conjugate-antibody complexes are formed. The antibody and conjugate may be added in any order to the test sample. The antibody may be immobilized on a solid support or a microparticle to facilitate separation of analyte-antibody complexes and conjugate-antibody complexes from the mixture.

Suitable methods for immobilizing the antibody on the solid support or microparticle include ionic, hydrophobic, covalent interactions, and the like. The solid support or microparticle can be chosen for its intrinsic ability to attract and immobilize the antibody. Alternatively, the solid support or microparticle may comprise an additional receptor that has the ability to attract and immobilize the antibody. The additional receptor may include a charged substance that is oppositely charged with respect to the antibody, which is immobilized (attached to) the solid support or microparticle and has the ability to immobilize the capture antibody through a specific binding reaction. The additional receptor enables the indirect binding of the capture antibody to the solid support or microparticle before the performance of the method or during the performance of the method.

After separation of the complexes from the mixture, the oxidant, basic solution, and zwitterionic detergent may be added to either the remaining mixture, which contains free or unbound conjugate, or the complexes, which contain bound conjugate. Either may be correlated to the amount of the analyte in the test sample. Upon formation of the composition, the chemiluminescent signal is generated and then detected. The chemiluminescent signal is compared to the reference standard and/or standard curve to quantify the amount of the analyte in the test sample.

b. Nucleic Acid Amplification and/or Hybridization

The method may be used in one or more assays that employ amplification and/or hybridization of nucleic acids. In such assays, the analyte is a target sequence and the compound of formula (I) is conjugated to the agent (e.g., a polynucleotide) as described above. The polynucleotide conjugated to the compound of formula (I) may be a primer or a probe.

(1) Primer

When the polynucleotide is a primer, the compound of formula (I) is linked to the 5′ hydroxy of the primer during primer synthesis. Thus, the compound of formula (I) is incorporated into an amplicon by virtue of the primer annealing to the target sequence in the test sample and being extended by an enzyme such as a DNA polymerase or a reverse transcriptase. Any primer not incorporated into an amplicon may be separated from the amplicons using routine techniques known to those skilled in the art. Addition of the oxidant, basic solution, and zwitterionic detergent to the test sample forms the mixture of the composition and test sample, and the chemiluminescent signal is generated and then detected. The detected chemiluminescent signal indicates the presence of the amplicon and thus the target sequence in the test sample.

The amplicon may further be immobilized on a solid support or a microparticle to facilitate separation of the amplicon from the test sample prior to or concurrently with detection of the chemiluminescent signal. Particularly, the amplicon may also include a specific binding member when the primer is a first primer of a primer pair. A second primer of the primer pair may be labeled with the specific binding member, and thus the specific binding member is incorporated into the amplicon by extending the second primer of the primer pair. Accordingly, the amplicon can be immobilized on the solid support or microparticle using the specific binding member and directly detected by virtue of the compound of formula (I).

(2) Probe

The polynucleotide conjugated to the compound of formula (I) may be a probe. The probe specifically binds (hybridizes to) the target sequence to form a hybrid of the probe and the target sequence. The probe is used in molar excess amounts relative to the maximum amount of the target sequence expected in the test sample. Any probe not bound to the target sequence may be separated from the hybrid of the probe and target sequence using routine techniques known to those skilled in the art. Upon formation of the mixture of the composition and test sample, the chemiluminescent signal is generated, and then detected. The measured intensity of the chemiluminescent signal may be compared to the reference standard and/or standard curve to quantify the amount of the target sequence in the test sample.

The hybrid of the probe and target sequence may further be immobilized on a solid support or a microparticle to facilitate separation of the hybrid from the test sample prior to or concurrently with detection of the chemiluminescent signal. A specific binding member may be incorporated into the target sequence, for example, a target sequence generated by extending a primer labeled with the specific binding member. Accordingly, the hybrid can be immobilized on the solid support or microparticle using the specific binding member and directly detected by virtue of the compound of formula (I).

The present invention has multiple aspects, illustrated by the following non-limiting examples.

### 4. EXAMPLES

### Example 1

### Effect of Detergent on Chemiluminescent Signal

The detergents Triton X-100 and N-dodecyl-N,N-dimethyl-3-ammonio-1-propanesulfonate (under the trade name Zwittergent 3-12) were compared to one another with regards to generation of a chemiluminescent signal. An immunoassay that used chemiluminescence in the detection of an analyte in a sample was used for this comparison of Triton X-100 and N-dodecyl-N,N-dimethyl-3-ammonio-1-propanesulfonate. In particular, two immunoassay formats were used in the comparison of Triton X-100 and N-dodecyl-N,N-dimethyl-3-ammonio-1-propanesulfonate, namely a competitive binding assay and a sandwich assay. Both the competitive binding and sandwich assay used antibodies that bound the analyte and an acridinium based compound for generation of the chemiluminescent signal.

Competitive Binding Assay

In the competitive binding assay, the acridinium based compound 3-(9-((3-carboxypropyl)(tosyl)carbamoyl)acridin-10-ium-10-yl)propane-1-sulfonate was conjugated to the analyte. The resulting conjugate was added to the sample and thus, competed with the analyte present in the sample for binding to the antibodies. Accordingly, a reaction vessel used in the competitive binding assay contained a mixture of the sample, conjugate, and antibodies that bound the analyte. The reaction vessel was used with the immunoassay analyzer ARCHITECT i2000SR sold by Abbott Diagnostics.

Two reaction vessels, receiving equivalent amounts of sample, conjugate, and antibodies, were used in the competitive binding assay. The first reaction vessel received a solution of hydrogen peroxide, 4.3 mM Triton X-100, a metal ion chelator, and nitric acid in water. Hydrogen peroxide is an oxidant as described above. The first reaction vessel then received a solution of sodium hydroxide and 32 mM Triton X-100 in water. Sodium hydroxide is a base, and thus, the solution containing sodium hydroxide is a basic solution as described above.

The second reaction vessel received a solution of hydrogen peroxide, 4.3 mM N-dodecyl-N,N-dimethyl-3-ammonio-1-propanesulfonate, a metal ion chelator, and nitric acid in water. The second reaction vessel then received a solution of sodium hydroxide and 32 mM N-dodecyl-N,N-dimethyl-3-ammonio-1-propanesulfonate in water.

Accordingly, the first and second reaction vessels received the same solutions with the only difference being the identity of the detergent (i.e., the first reaction vessel received solutions containing Triton X-100 and the second reaction vessel received solutions containing N-dodecyl-N,N-dimethyl-3-ammonio-1-propanesulfonate).

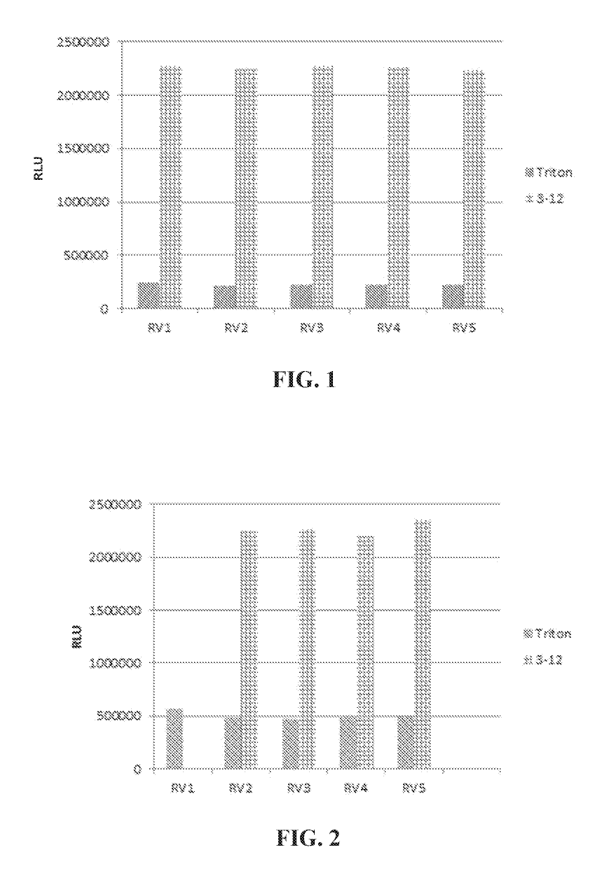

The chemiluminescence generated in the first and second reaction vessels was measured and integrated by the immunoassay analyzer ARCHITECT i2000SR (Abbott Diagnostics). This experiment was done in quintuplicate, i.e., five pairs of reaction vessels (RV), (RV1, RV2, RV3, RV4, and RV5). The resulting relative light units (RLU) for RV1, RV2, RV3, RV4, and RV5 are shown in FIG. 1. In FIG. 1, for each of RV1, RV2, RV3, RV4, and RV5, the solid bar represented the signal from the reaction vessel receiving Triton X-100 while the cross-hatched bar bar represented the signal from the reaction vessel receiving N-dodecyl-N,N-dimethyl-3-ammonio-1-propanesulfonate.

These data showed that N-dodecyl-N,N-dimethyl-3-ammonio-1-propanesulfonate significantly enhanced the chemiluminescent signal as compared to Triton X-100. Specifically, the chemiluminescent signal was 10-fold higher when N-dodecyl-N,N-dimethyl-3-ammonio-1-propanesulfonate was used in the solutions as compared to Triton X-100. Accordingly, solutions containing N-dodecyl-N,N-dimethyl-3-ammonio-1-propanesulfonate enhanced the chemiluminescent signal by 10-fold over the chemiluminescent signal generated when Triton X-100 was used in the solutions. The combination of the acridinium based compound 3-(9-((3-carboxypropyl)(tosyl)carbamoyl)acridin-10-ium-10-yl)propane-1-sulfonate and N-dodecyl-N,N-dimethyl-3-ammonio-1-propanesulfonate provided an enhanced chemiluminescent signal (i.e., 10-fold) as compared to the combination of the acridinium based compound 3-(9-((3-carboxypropyl)(tosyl)carbamoyl)acridin-10-ium-10-yl)propane-1-sulfonate and Triton X-100, which is not a zwitterionic detergent.

Sandwich Assay

In the sandwich assay, the acridinium based compound 3-(9-(((4-(3-carboxypropyl)phenyl)sulfonyl)(3-sulfopropyl)carbamoyl)acridin-10-ium-10-yl)propane-1-sulfonate was conjugated to an antibody that bound the analyte. The resulting conjugate was added to the sample, along with particles that were coupled to antibodies that also bound the analyte. Accordingly, the analyte present in the test sample was bound by both antibody coupled to the particle and antibody conjugated to the acridinium based compound 3-(9-(((4-(3-carboxypropyl)phenyl)sulfonyl)(3-sulfopropyl)carbamoyl)acridin-10-ium-10-yl)propane-1-sulfonate, and thus, was sandwiched between these antibodies.

A reaction vessel used in the sandwich assay thus contained a mixture of the sample, conjugate, and particles. The reaction vessel was used with the immunoassay analyzer ARCHITECT i2000SR sold by Abbott Diagnostics.

Two reaction vessels, receiving equivalent amounts of sample, conjugate, and particles, were used in the sandwich binding assay. The first reaction vessel received a solution of hydrogen peroxide, 4.3 mM Triton X-100, a metal ion chelator, and nitric acid in water. The first reaction vessel then received a solution of sodium hydroxide and 32 mM Triton X-100 in water.

The second reaction vessel received a solution of hydrogen peroxide, 4.3 mM N-dodecyl-N,N-dimethyl-3-ammonio-1-propanesulfonate, a metal ion chelator, and nitric acid in water. The second reaction vessel then received a solution of sodium hydroxide and 32 mM N-dodecyl-N,N-dimethyl-3-ammonio-1-propanesulfonate in water.

Accordingly, the first and second reaction vessels received the same solutions with the only difference being the identity of the detergent (i.e., the first reaction vessel received solutions containing Triton X-100 and the second reaction vessel received solutions containing N-dodecyl-N,N-dimethyl-3-ammonio-1-propanesulfonate).

The chemiluminescence generated in the first and second reaction vessels was measured and integrated by the immunoassay analyzer ARCHITECT i2000SR (Abbott Diagnostics). This experiment was done in quintuplicate, i.e., five pairs of reaction vessels (RV), (RV1, RV2, RV3, RV4, and RV5). The resulting relative light units (RLU) for RV1, RV2, RV3, RV4, and RV5 are shown in FIG. 2. In FIG. 2, for each of RV1, RV2, RV3, RV4, and RV5, the solid bar represented the signal from the reaction vessel receiving Triton X-100 while the cross-hatched bar represented the signal from the reaction vessel receiving N-dodecyl-N,N-dimethyl-3-ammonio-1-propanesulfonate.

These data showed that N-dodecyl-N,N-dimethyl-3-ammonio-1-propanesulfonate significantly enhanced the chemiluminescent signal as compared to Triton X-100. Specifically, the chemiluminescent signal was 5-fold higher when N-dodecyl-N,N-dimethyl-3-ammonio-1-propanesulfonate was used in the solutions as compared to Triton X-100. Accordingly, solutions containing N-dodecyl-N,N-dimethyl-3-ammonio-1-propanesulfonate enhanced the chemiluminescent signal by 5-fold over the chemiluminescent signal generated when Triton X-100 was used in the solutions. The combination of the acridinium based compound 3-(9-(((4-(3-carboxypropyl)phenyl)sulfonyl)(3-sulfopropyl)carbamoyl)acridin-10-ium-10-yl)propane-1-sulfonate and N-dodecyl-N,N-dimethyl-3-ammonio-1-propanesulfonate provided an enhanced chemiluminescent signal (i.e., 5-fold) as compared to the combination of the acridinium based compound 3-(9-(((4-(3-carboxypropyl)phenyl)sulfonyl)(3-sulfopropyl)carbamoyl)acridin-10-ium-10-yl)propane-1-sulfonate and Triton X-100, which is not a zwitterionic detergent.

### Example 2

### Effect of Concentration on Chemiluminescent Signal

As described above in Example 1, the combination of the acridinium based compound 3 -(9-(((4-(3 -carboxypropyl)phenyl)sulfonyl)(3-sulfopropyl)carbamoyl)acridin-10-ium-10-yl)propane-1-sulfonate and zwitterionic detergent provided enhanced chemiluminescence as compared to the combination of the acridinium based compound 3-(9-(((4-(3-carboxypropyl)phenyl)sulfonyl)(3-sulfopropyl)carbamoyl)acridin-10-ium-10-yl)propane-1-sulfonate and a non-zwitterionic detergent. To further examine this enhanced chemiluminescence, different concentrations of the zwitterionic detergent were employed in the sandwich assay described above in Example 1.

Specifically, three reaction vessels, receiving equivalent amounts of sample, conjugate, and particles, were used in the sandwich assay. The first reaction vessel received Triton X-100 while the second and third reaction vessels received different concentrations of the zwitterionic detergent 3-(Tetradecyldimethylammonio)propanesulfonate (under the trade name ZWITTERGENT 3-14).

The first reaction vessel received a solution of hydrogen peroxide, 4.3 mM Triton X-100, a metal ion chelator, and nitric acid in water. The first reaction vessel then received a solution of sodium hydroxide and 32 mM Triton X-100 in water.

The second reaction vessel received a solution of hydrogen peroxide, 0.6 mM 3-(Tetradecyldimethylammonio)propanesulfonate, a metal ion chelator, and nitric acid in water. The second reaction vessel then received a solution of sodium hydroxide and 0.6 mM 3-(Tetradecyldimethylammonio)propanesulfonate in water.

The third reaction vessel received a solution of hydrogen peroxide, 6 mM 3-(Tetradecyldimethylammonio)propanesulfonate, a metal ion chelator, and nitric acid in water. The third reaction vessel then received a solution of sodium hydroxide and 0.6 mM 3-(Tetradecyldimethylammonio)propanesulfonate in water.

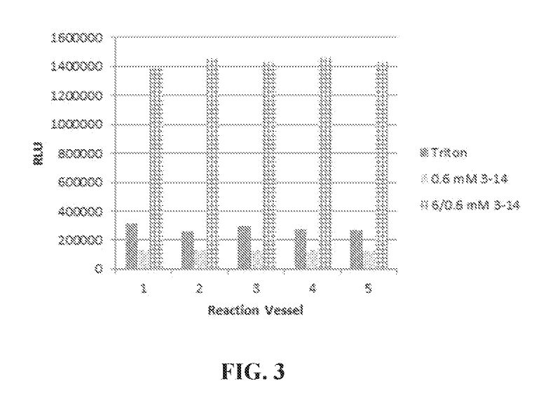

The chemiluminescence generated in the first, second, and third reaction vessels was measured and integrated by the immunoassay analyzer ARCHITECT i2000SR (Abbott Diagnostics). This experiment was done in quintuplicate, i.e., five groups of reaction vessels (RV), (RV1, RV2, RV3, RV4, and RV5). The resulting relative light units (RLU) for RV1, RV2, RV3, RV4, and RV5 are shown in FIG. 3. In FIG. 3, for each of RV1, RV2, RV3, RV4, and RV5, the solid dark bar represented the signal from the reaction vessel receiving Triton X-100 while the solid light bar and the cross-hatched bar represented the signal from the reaction vessel receiving 0.6 mM/0.6 mM 3-(Tetradecyldimethylammonio)propanesulfonate and the signal from the reaction vessel receiving 0.6 mM/6 mM 3-(Tetradecyldimethylammonio)propanesulfonate, respectively.

As observed in FIGS. 1 and 2 and described above in Example 1, the zwitterionic detergent enhanced the chemiluminescent signal as compared to Triton X-100, but in a concentration dependent manner. In particular, a concentration of 0.6 mM of 3-(Tetradecyldimethylammonio)propanesulfonate in both the hydrogen peroxide and sodium hydroxide solutions did not provide an enhanced or increased chemiluminescent signal as compared to Triton X-100. However, when the concentration of 3-(Tetradecyldimethylammonio)propanesulfonate was increased to 6 mM in the first solution (i.e., the solution with hydrogen peroxide), the resulting chemiluminescent signal was significantly enhanced relative to the chemiluminescent signal observed when Triton X-100 or 0.6 mM 3-(Tetradecyldimethylammonio)propanesulfonate was used in the both the hydrogen peroxide and sodium hydroxide solutions. Accordingly, the concentration of the zwitterionic detergent may be used to control or alter the amount of the light produced in a chemiluminescent reaction with the acridinium based compound 3-(9-(((4-(3-carboxypropyl)phenyl)sulfonyl)(3-sulfopropyl)carbamoyl)acridin-10-ium-10-yl)propane-1-sulfonate.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1.-21. (canceled)

22. A method for detecting an analyte in a test sample, the method comprising the step of:
contacting the sample with a composition comprising
(a) compound of formula (I) 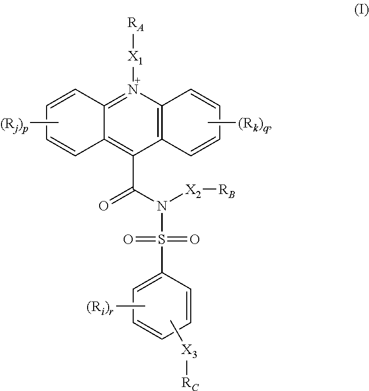
wherein
X₁, X₂ and X₃ are each independently selected from a bond and alkylene;
one of R_{A} and R_{B} is —SO₃⁻;
the other of R_{A} and R_{B} is selected from the group consisting of hydrogen, alkyl, alkenyl, alkynyl, aryl, amino, amido, alkoxy, hydroxyl, carboxy, halogen, halide, nitro, cyano, —SO₃H and —C(O)R^{1a};
R_{C} is —C(O)R^{2a} or H;
Rⱼ, Rₖ and Rᵢ, at each occurrence, are independently selected from the group consisting of hydrogen, alkyl, alkenyl, alkynyl, aryl, arylalkyl, amino, amido, acyl, alkoxy, hydroxyl, carboxy, halogen, halide, nitro, cyano, —SO₃H, —SO₃⁻, sulfoalkyl, carboxyalkyl and acylalkyl;
p, q and r are each independently 0, 1, 2, 3 or 4; and
R^{1a} and R^{2a}, at each occurrence, are independently selected from the group consisting of hydrogen, alkyl, alkoxy, haloalkyl, haloalkyloxy, hydroxyl, cycloalkyl, cycloalkyloxy, cycloalkylalkyl, aryl, aryloxy, arylalkyl, halogen, heterocycle, heterocycleoxy, heterocyclealkyl, heteroaryl, heteroaryloxy and heteroarylalkyl; and
(b) N-dodecyl-N,N-dimethyl-3-ammonio-1-propanesulfonate
wherein contacting the test sample with the composition forms a mixture.

23. The method of claim 22, wherein
X₃ is alkylene;
one of R_{A} and R_{B} is —SO₃⁻; and
the other of R_{A} and R_{B} is selected from the group consisting of alkyl, hydroxyl, carboxy, —SO₃H and —C(O)R^{1a}.

24. The method of claim 22, wherein
X₃ is —CH₂—CH₂—CH₂—; and
R^{2a} is selected from the group consisting of hydroxyl, aryloxy, heterocycleoxy and heteroaryloxy.

25. The method of claim 22, wherein
X₁ and X₂ are alkylene;
one of R_{A} and R_{B} is —SO₃⁻; and
the other of R_{A} and R_{B} is —SO₃H.

26. The method of claim 22, wherein
R^{2a} is selected from the group consisting of hydroxyl, 4-nitrophenoxy, perchlorophenoxy, perfluorophenoxy, 2,5-dioxopyrrolidin-1-yloxy, 1,3-dioxoisoindolin-2-yloxy, and benzotriazol-1-yloxy.

27. The method of claim 22, wherein the compound of Formula (I) is selected from the group consisting of:
3-(9-(((4-(3-carboxypropyl)phenyl)sulfonyl)(3-sulfopropyl)carbamoyl)acridin-10-ium-10-yl)propane-1-sulfonate;
3-(9-((3-carboxypropyl)(tosyl)carbamoyl)acridin-10-ium-10-yl)propane-1-sulfonate;
3-(9-(((4-(4-oxo-4-(perfluorophenoxy)butyl)phenyl)sulfonyl)(3-sulfopropyl)carbamoyl)acridin-10-ium-10-yl)propane-1-sulfonate;
3-(N-((4-(3-carboxypropyl)phenyl)sulfonyl)-10-(3-sulfopropyl)acridin-10-ium-9-carboxamido)propane-1-sulfonate; and
3-(N-((4-(4-oxo-4-(perfluorophenoxy)butyl)phenyl)sulfonyl)-10-(3-sulfopropyl)acridin-10-ium-9-carboxamido)propane-1-sulfonate.

28. The method of claim 22, wherein the N-dodecyl-N,N-dimethyl-3-ammonio-1-propanesulfonate is present in an amount sufficient to form micelles.

29. The method of claim 22, wherein the mixture is allowed to incubate for a period of time from about 1 second to about 18 minutes.

30. The method of claim 22, further comprising pretreating the test sample prior to contacting the sample with the composition.

31. The method of claim 30, wherein the pretreatment is selected from the group consisting of dilution, ultrafiltration, extraction, precipitation, dialysis, chromatography, and digestion.

32. The method of claim 22, further comprising the steps of:
generating a chemiluminescent signal;
detecting the chemiluminescent signal; and
measuring the chemiluminescent signal.

33. The method of claim 32, wherein the mixture is formed and the signal is detected concurrently.

34. The method of claim 32, wherein the duration of the detection ranges from about 0.01 seconds to about 360 seconds.

35. The method of claim 32, wherein the detection is semi-automated.

36. The method of claim 32, wherein the detection is automated.

37. The method of claim 32, wherein the chemiluminescent signal is related to an amount of the analyte in the test sample.

38. The method of claim 37, wherein the amount of the analyte in the test sample is proportional to the intensity of the signal generated.

39. The method of claim 37, wherein the amount of analyte in the test sample is inversely proportional to the signal generated.

40. The method of claim 37, wherein the amount of analyte is quantified by comparing an amount of light generated to a standard curve for the analyte.

41. The method of claim 22, wherein the analyte is detected by an immunoassay.
